(19) United States
(12) Patent Application Publication
(10) Pub. No.: US 2023/0381008 A1
(43) Pub. Date: Nov. 30, 2023

(54) Fecal Guard Device

(71) Applicant: Joan L. Williams, Pittsburgh, PA (US)

(72) Inventor: Joan L. Williams, Pittsburgh, PA (US)

(21) Appl. No.: 18/310,263

(22) Filed: May 1, 2023

Related U.S. Application Data
(60) Provisional application No. 63/346,006, filed on May 26, 2022.

(51) Int. Cl. A61F 5/451

(52) U.S. Cl. CPC A61F 5/451

(57) ABSTRACT

The present invention relates to a novel fecal guard device. The device is a disposable, sanitary fecal matter guard for patients who no longer meet FDA approved guidelines for an indwelling fecal management catheter. The guard device prevents fecal matter from infiltrating a stage 4 wound on the sacrum during healing. The fecal guard device comprises a body component shaped as a funnel with small gauze covered foam insert within the hollow opening. The body component is placed near the sacrum wound, cleaned according to medical guidelines as recommended by the medical team. The wound is covered with gauze and anti-fungal cream. The anus is covered with gauze and anti-fungal cream or powder to preserve integrity of the skin. The body component is secured via gauze tape onto gauze covering near sacrum wound. The body component acts as a shield, leaving wound on sacrum untouched by fecal matter.

### CROSS-REFERENCE TO RELATED APPLICATION

The present application claims priority to, and the benefit of, U.S. Provisional Application No. 63/346,006, which was filed on May 26, 2022, and is incorporated herein by reference in its entirety.

### FIELD OF THE INVENTION

The present invention relates generally to the field of fecal guard devices. More specifically, the present invention relates to a disposable wound treatment kit for stage 4 sacrum wounds capable of reducing the chance of infection. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in fecal guard devices. Generally, stage 4 sacrum wounds are often susceptible through fecal matter, requiring exceptional medical care to prevent. Sepsis, osteomyelitis, gangrene, and other conditions can occur if the wound and its dressing are not properly maintained. Further, skin breakdown can also occur near the anal area due to excess moisture and bacteria from fecal matter. These types of wounds often result in slow healing and are very uncomfortable for a patient.

Accordingly, a need remains for a fecal guard device that is disposable and sanitary and acts as a guard for patients who no longer meet FDA (Food and Drug Administration) guidelines to continue with an indwelling fecal management catheter which is up to 29 days. Further, a fecal guard device is necessary to prevent fecal matter from infiltrating a stage 4 wound on the sacrum during its healing process. The fecal guard device would act as a shield, leaving the wound on the sacrum untouched by any fecal matter. Further, the fecal guard device can also be available as a kit, with gauze, anti-fungal cream, and powder, etc.

Therefore, there exists a long-felt need in the art for a fecal guard device that provides users with a disposable wound treatment kit for stage 4 sacrum wounds capable of reducing the chance of infection. There is also a long-felt need in the art for a fecal guard device that features multiple gauze pads, anti-fungal powder and cream, and a small funnel with a small foam insert that work in conjunction to prevent, as well as ensure, the wound is untouched by fecal matter. Further, there is a long-felt need in the art for a fecal guard device that allows caregivers to wipe away fecal matter without having to constantly change the wound dressing, maintaining coverage and protection of the wound. Moreover, there is a long-felt need in the art for a device that promotes faster and more effective healing, protecting the skin around the anal area and offering an alternative to having a colostomy procedure. Further, there is a long-felt need in the art for a fecal guard device that allows patients to expel fecal matter without worry of the fecal matter touching the wound. Finally, there is a long-felt need in the art for a fecal guard device that allows users to wipe away fecal matter without interrupting the scheduled wound dressing change.

The subject matter disclosed and claimed herein, in one embodiment thereof, comprises a fecal guard device. The device is a disposable and sanitary fecal matter guard for patients who no longer meet FDA guidelines to have an indwelling fecal management catheter. (The FDA approved bowel management systems for up to 29 days for the diversion of stool into the collection system.) The guard device prevents fecal matter from infiltrating a stage 4 wound on the sacrum during its healing process. The fecal guard device comprises a body component shaped as a funnel with a small foam insert within the hollow opening. The body component is placed near the sacrum wound, which is covered with gauze and anti-fungal cream. The anus of a patient is also covered with gauze and anti-fungal cream or powder to help preserve the integrity of the skin. The body component is then secured via gauze tape onto this gauze covering near the sacrum wound. The body component acts as a shield leaving the wound on the sacrum untouched by any fecal matter. Thus, patients can expel fecal matter without worry of the fecal matter touching the sacrum wound. Users can wipe away fecal matter without interrupting the scheduled wound dressing change. The wound is left clean and untouched for optimal healing. In one embodiment, the fecal guard device is available as a kit, including gauze, anti-fungal cream, and powder.

In this manner, the fecal guard device of the present invention accomplishes all of the foregoing objectives and provides users with a device that prevents fecal matter from contacting a stage 4 wound on the sacrum. The device is a shield that is placed near the sacrum wound to leave the wound clean. The device can be manufactured as a kit with gauze and anti-fungal cream or powder.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a fecal guard device. The device is a disposable and sanitary fecal matter guard for those patients who no longer meet FDA guidelines for an indwelling fecal management catheter. The guard device prevents fecal matter from infiltrating a stage 4 wound on the sacrum during its healing process. The fecal guard device comprises a body component shaped as a funnel with a small foam insert within the hollow opening, wherein gauze taping secures the small foam insert within the funnel body component. The sacrum wound is cleaned according to medical recommendations and the surrounding skin area is covered with gauze and anti-fungal cream. The anus of a patient is also covered with gauze and anti-fungal cream or powder to help preserve the integrity of the skin. The funnel body component is then secured on this gauze via a friction fit with the user's gluteus maximus, with the narrower end near the sacrum wound.

In one embodiment, the fecal guard device comprises a body component shaped as a funnel. Specifically, the body component is a hollow, cone-shaped funnel having a first end and an open second end, wherein the first end is tapered down to a point and the second end remains wide and open.

In one embodiment, a small gauze covered foam insert component is inserted into the open second end. The small covered gauze foam insert is shaped and sized to fill the open second end and retained within the second end during use. Further, the small foam insert would be secured within the second end via gauze tape, adhesive, or any other suitable securing means as is known in the art.

In one embodiment, the body component is a cone-shaped funnel that is solid instead of hollow and does not include the foam insert. In either embodiment, the funnel body component is inserted on the gauze padding over a user's anus and friction fit between a user's buttocks.

In one embodiment, the fecal guard device is available as a kit. As a kit, it includes not only the funnel body component, but also multiple gauze pads of varying shapes and sizes, anti-fungal cream and powder, gauze tape, etc., or any other suitable supplies as is known in the art. The kit can also be stored in a container with a sterile-packed lid or be terminally sterilized in a container for storage.

In one embodiment, the sacrum wound is covered with a gauze pad and anti-fungal cream and/or powder. Generally, the gauze pad is a 4×4 square pad, but can be any suitable size and shape as is known in the art. The wound dressing is applied according to general medical recommendations.

In one embodiment, the anus of a patient or user is also covered with a gauze pad and anti-fungal cream and/or powder to help preserve the integrity of the skin in this area. Generally, the gauze pad is a 4×4 square pad, but can be any suitable size and shape as is known in the art. The wound dressing is applied according to general medical recommendations.

In operation, the closed first end (or tip) of the funnel body component is positioned near the user's sacrum wound and the open second end and foam insert is positioned by the user's anus to act as a shield preventing fecal matter from infiltrating the stage 4 wound on the sacrum during its healing process. Thus, the funnel body component is inserted on the gauze padding over a user's anus and friction fit between a user's gluteus maximus during use. The funnel body component then acts as a shield, leaving the wound on the sacrum untouched by any fecal matter. Thus, patients can expel fecal matter without worry of the fecal matter touching the sacrum wound. Users can wipe away fecal matter without interrupting the scheduled wound dressing change. The wound is left clean and untouched for optimal healing.

In one embodiment, the funnel body component can be manufactured from a non-foam elastomer, thermoplastic polyurethanes, thermoplastic polyolefins, polyamides, polyurea, polyesters, polyethylene, and other suitable polymer materials as is known in the art.

In yet another embodiment, the fecal guard device comprises a plurality of indicia.

In yet another embodiment, a method of preventing fecal matter from infiltrating a stage 4 wound on the sacrum during its healing process is disclosed. The method includes the steps of providing a fecal guard device comprising a funnel body component with a small gauze covered foam insert within the hollow opening. The method also comprises covering the anus of a patient with a gauze pad and anti-fungal cream and/or powder. Further, the method comprises covering the sacrum wound with a gauze pad and anti-fungal cream and/or powder. The method comprises securing the funnel body component to the gauze pad on the anus near the sacrum wound via gauze tape. The method also comprises expelling fecal matter without worry of the fecal matter touching the sacrum wound. Finally, the method comprises wiping away fecal matter without interrupting a scheduled sacrum wound dressing change.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a top perspective view of one embodiment of the fecal guard device of the present invention showing the device available as a kit in accordance with the disclosed architecture;

FIG. 2 illustrates a top perspective view of one embodiment of the fecal guard device of the present invention showing the kit stored in a container in accordance with the disclosed architecture;

FIG. 3 illustrates a top perspective view of one embodiment of the fecal guard device of the present invention showing the gauze pad with anti-fungal powder and cream applied near the sacrum wound area in accordance with the disclosed architecture;

FIG. 4 illustrates a top perspective view of one embodiment of the fecal guard device of the present invention showing the funnel body component with the foam insert applied over the gauze pad in accordance with the disclosed architecture;

FIG. 5 illustrates a top perspective view of one embodiment of the fecal guard device of the present invention showing the gauze pad applied to the sacrum wound to prevent fecal matter from reaching the wound in accordance with the disclosed architecture; and

FIG. 6 illustrates a flowchart showing the method of preventing fecal matter from infiltrating a stage 4 wound on the sacrum during its healing process in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a fecal guard device that provides users with a disposable wound treatment kit for stage 4 sacrum wounds capable of reducing the chance of infection. There is also a long-felt need in the art for a fecal guard device that features multiple gauze pads, anti-fungal powder and cream, and a small funnel with a small foam insert that work in conjunction to prevent, as well as ensure, the wound in untouched by fecal matter. Further, there is a long-felt need in the art for a fecal guard device that allows caregivers to wipe away fecal matter without having to constantly change the wound dressing, maintaining coverage and protection of the wound. Moreover, there is a long-felt need in the art for a device that promotes faster and more effective healing, protecting the skin around the anal area and offering an alternative to having a colostomy procedure. Further, there is a long-felt need in the art for a fecal guard device that allows patients to expel fecal matter without worry of the fecal matter touching the wound. Finally, there is a long-felt need in the art for a fecal guard device that allows users to wipe away fecal matter without interrupting the scheduled wound dressing change.

The present invention, in one exemplary embodiment, is a novel fecal guard device. The device is a disposable and sanitary fecal matter guard for patients who are no longer meeting FDA approved for an indwelling fecal management catheter. Specifically, CDC guidelines states “A stool management system cannot be used for more than 29 days.” Accordingly, patients with stage 4 sacrum wounds are likely to become contaminated with incontinent stool and experience other debilitating issues. The guard device prevents fecal matter from infiltrating a stage 4 wound on the sacrum during its healing process. The fecal guard device comprises a body component shaped as a funnel with a small gauze covered foam insert within the hollow opening. The funnel body component is placed near the sacrum wound, which is covered with gauze and anti-fungal cream. The anus of a patient is also covered with gauze and anti-fungal cream or powder to help preserve the integrity of the skin. The funnel body component is then secured via gauze tape onto this gauze covering, near the sacrum wound. The funnel body component acts as a shield, leaving the wound on the sacrum untouched by any fecal matter. The present invention also includes a novel method of preventing fecal matter from infiltrating a stage 4 wound on the sacrum during its healing process. The method includes the steps of providing a fecal guard device comprising a funnel body component with a small foam insert within the hollow opening. The method also comprises covering the anus of a patient with a gauze pad and anti-fungal cream and/or powder. Further, the method comprises covering the sacrum wound with a gauze pad and anti-fungal cream and/or powder. The method comprises securing the funnel body component to the gauze pad on the anus, near the sacrum wound via gauze tape. The method also comprises expelling fecal matter without worry of the fecal matter touching the sacrum wound. Finally, the method comprises wiping away fecal matter without interrupting a scheduled sacrum wound dressing change.

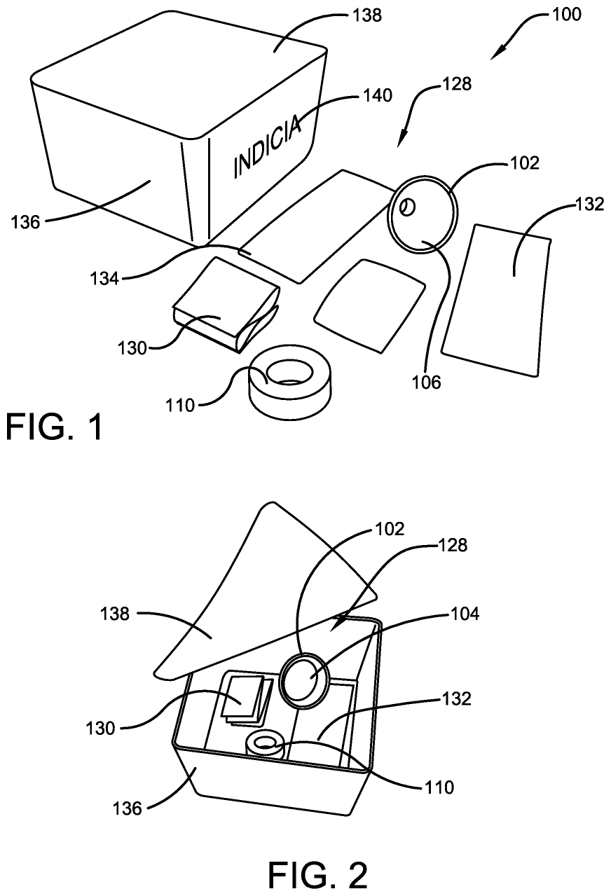

Referring initially to the drawings, FIG. 1 illustrates a perspective view of one embodiment of the fecal guard device 100 of the present invention. In the present embodiment, the fecal guard device 100 is a disposable and sanitary fecal matter guard for patients who are no longer meeting FDA approved guidelines for an indwelling fecal management catheter. The improved fecal guard device 100 acts as a shield, preventing fecal matter from infiltrating a stage 4 wound on the sacrum during its healing process, thus leaving a sacrum wound 108 untouched by any fecal matter. Specifically, the fecal guard device 100 comprises a body component 102 shaped as a funnel with a small foam insert 104 within the hollow opening 106, wherein gauze taping 110 secures the small foam insert 104 within the funnel body component 102. The sacrum wound 108 is covered with gauze and anti-fungal cream. The anus 112 of a patient 114 is also covered with gauze and anti-fungal cream or powder to help preserve the integrity of the skin 116. The funnel body component 102 is then secured on this gauze via a friction fit with the user's gluteus maximus 118, with the narrower end near the sacrum wound 108.

In one embodiment, the fecal guard device 100 comprises a body component 102 shaped as a funnel. Specifically, the body component 102 is a hollow, cone-shaped funnel 120 having a first end 122 and an open second end 124, wherein the first end 122 is tapered down to a point 126 and the second end 124 remains wide and open. However, the body component 102 does not have to be funnel-shaped and can be any suitable shape as is known in the art, such as oval, cylindrical, square, rectangular, etc., depending on the needs and/or wants of a user 114.

Further, a small foam insert component 104 is inserted into the open second end. The small foam insert component 104 is shaped and sized to fill the open second end 124 and be retained within the second end 124 during use. Further, the small foam insert component 104 would be secured within the second end 124 via gauze tape 110, adhesive, or any other suitable securing means as is known in the art, depending on the wants and/or needs of a user. Additionally, the small foam insert component 104 can be shaped and sized to be friction fit within the second end 124, as well. If friction fit, then the small foam insert component 104 would not require additional securing means to stay positioned within the second end 124, such as gauze tape 110, adhesive, etc.

In another embodiment, the body component 102 is a cone-shaped funnel 120 that is solid instead of hollow and does not include the foam insert component 104. In either embodiment, the funnel body component 102 is inserted on the gauze padding over a user's anus 112 and friction fit between a user's gluteus maximus 118 during use. Furthermore, the body component 102 does not have to be funnel-shaped and can be any suitable shape as is known in the art, such as oval, cylindrical, square, rectangular, etc., depending on the needs and/or wants of a user 114. However, no matter the shape, the body component 102, in this embodiment, would be solid instead of hollow, thus not requiring the foam insert component 104.

Typically, the funnel body component 102 and/or the foam insert component 104 can be manufactured from a non-foam elastomer, thermoplastic polyurethanes, thermoplastic polyolefins, polyamides, polyurea, polyesters, polyethylene, and any other suitable polymer materials as is known in the art, depending on the wants and/or needs of a user. The funnel body component 102 and the foam insert component 104 can be manufactured of the same or different materials depending on the wants and/or needs of a user.

Further, the fecal guard device 100 comprises a plurality of indicia 140. The funnel body component 102 of the device 100 may include advertising, trademark, other letters, designs, or characters, printed, painted, stamped, or integrated into the funnel body component 102, or any other indicia 140 as is known in the art. Specifically, any suitable indicia 140 as is known in the art can be included, such as, but not limited to, patterns, logos, emblems, images, symbols, designs, letters, words, characters, animals, advertisements, brands, etc., that may or may not be wound, bandage, or brand related.

As shown in FIGS. 1-2, the fecal guard device 100 is available as a kit 128. As a kit 128, it includes not only the funnel body component 102, but also multiple gauze pads 130 of varying shapes and sizes, anti-fungal cream 132 and powder 134, gauze tape 110, etc., or any other suitable supplies as is known in the art, depending on the needs and/or wants of a user. The kit 128 can also be stored in a container 136 with a sterile-packed lid 138 or be terminally sterilized in a container 136 for storage. The container 136 can be any suitable shape and size as is known in the art as long as it can retain all components of the kit 128 securely within it.

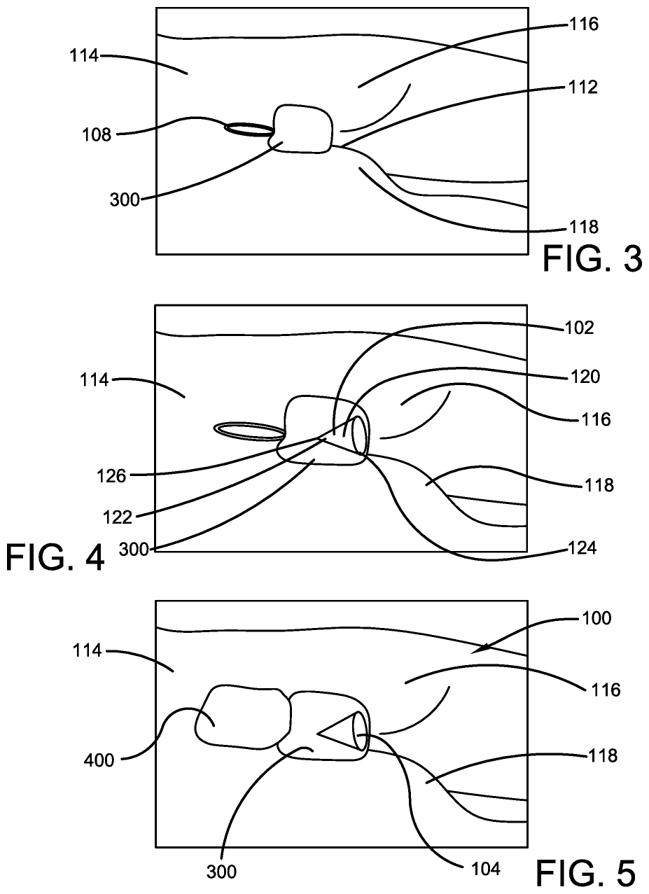

As shown in FIG. 3, the anus 112 of a patient or user 114 is also covered with a gauze pad 300 and anti-fungal cream 132 and/or powder 134 to help preserve the integrity of the skin 116 in this area. Generally, the gauze pad 300 is a 4×4 square pad but can be any suitable size and shape as is known in the art, depending on the needs and/or wants of a user. The wound dressing 300 is applied according to general medical recommendations.

As shown in FIGS. 4-5, the sacrum wound 108 is covered with a gauze pad 400 and anti-fungal cream 132 and/or powder 134. Generally, the gauze pad 400 is a 4×4 square pad but can be any suitable size and shape as is known in the art, depending on the needs and/or wants of a user. The wound dressing 400 is applied according to general medical recommendations.

In operation, the closed first end 122 (or tip 126) of the funnel body component 102 is positioned near the user's sacrum wound 108 and the open second end 124 and foam insert 104 is positioned by the user's anus 112 to act as a shield preventing fecal matter from infiltrating the stage 4 wound 108 on the sacrum during its healing process. Thus, the funnel body component 102 is inserted on the gauze padding 300 over a user's anus 112 and friction fit between a user's gluteus maximus 118 during use. The funnel body component 102 then acts as a shield leaving the wound 108 on the sacrum untouched by any fecal matter. Thus, patients 114 can expel fecal matter without worry of the fecal matter touching the sacrum wound 108. Further, users 114 can wipe away fecal matter without interrupting the scheduled wound dressing change. The wound 108 is left clean and untouched for optimal healing.

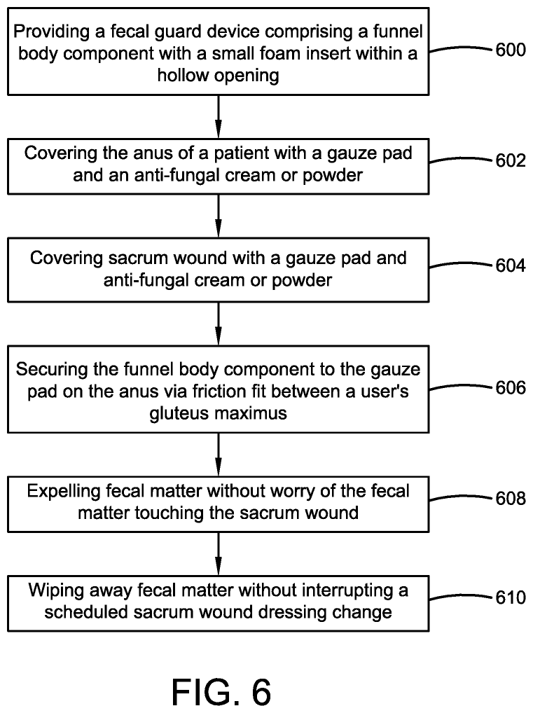

FIG. 6 illustrates a flowchart of the method of preventing fecal matter from infiltrating a stage 4 wound on the sacrum during its healing process. The method includes the steps of at 600, providing a fecal guard device comprising a funnel body component with a small foam insert within the hollow opening. The method also comprises at 602, covering the anus of a patient with a gauze pad and anti-fungal cream and/or powder. Further, the method comprises at 604, covering the sacrum wound with a gauze pad and anti-fungal cream and/or powder. The method comprises at 606, securing the funnel body component to the gauze pad on the anus via friction fit between a user's gluteus maximus. The method also comprises at 608, expelling fecal matter without worry of the fecal matter touching the sacrum wound. Finally, the method comprises at 610, wiping away fecal matter without interrupting a scheduled sacrum wound dressing change.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “fecal guard device”, “guard device”, “fecal device”, and “device” are interchangeable and refer to the fecal guard device 100 of the present invention.

Notwithstanding the foregoing, the fecal guard device 100 of the present invention can be of any suitable size and configuration as is known in the art without affecting the overall concept of the invention, provided that it accomplishes the above-stated objectives. One of ordinary skill in the art will appreciate that the fecal guard device 100 as shown in FIGS. 1-6 are for illustrative purposes only, and that many other sizes and shapes of the fecal guard device 100 are well within the scope of the present disclosure. Although the dimensions of the fecal guard device 100 are important design parameters for user convenience, the fecal guard device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A fecal guard device that acts as a shield, preventing fecal matter from infiltrating a stage 4 wound on the sacrum during its healing process, the fecal guard device comprising:
a body component;
wherein a sacrum wound is covered with a gauze;
wherein a user's anus is covered with a gauze;
wherein the body component is positioned on the gauze of the user's anus;
wherein the body component acts as a shield, leaving the sacrum wound untouched by any fecal matter; and
further wherein users can wipe away fecal matter without interrupting a scheduled wound dressing change for the sacrum wound.

2. The fecal guard device of claim 1, wherein the body component is a hollow, cone-shaped funnel.

3. The fecal guard device of claim 2, wherein the body component has a first end and an open second end, wherein the first end is tapered down to a point and the second end remains wide and open.

4. The fecal guard device of claim 3, wherein a small foam insert component is inserted into the open second end and secured.

5. The fecal guard device of claim 4, wherein the small foam insert component would be secured within the open second end via gauze tape.

6. The fecal guard device of claim 5, wherein the fecal guard device is available as a kit.

7. The fecal guard device of claim 6, wherein the kit includes the body component, multiple gauze pads of varying shapes and sizes, anti-fungal cream and powder, and gauze tape.

8. The fecal guard device of claim 7, wherein the kit is stored in a container with a sterile-packed lid.

9. The fecal guard device of claim 8, wherein the anus of a user is covered with a gauze pad and anti-fungal cream and powder to help preserve integrity of user's skin in this area.

10. The fecal guard device of claim 9, wherein the sacrum wound is covered with a gauze pad and anti-fungal cream and powder to prevent germs and aid healing.

11. The fecal guard device of claim 10, wherein in operation, the first end of the body component is positioned near the sacrum wound and the open second end and the small foam insert component is positioned by the anus to act as a shield, preventing fecal matter from infiltrating the sacrum wound during its healing process.

12. The fecal guard device of claim 11, wherein the body component is inserted on the gauze pad over the anus and friction fit between a user's gluteus maximus, during use.

13. The fecal guard device of claim 12 further comprising a plurality of indicia.

14. A fecal guard device that acts as a shield, preventing fecal matter from infiltrating a stage 4 wound on the sacrum during its healing process, the fecal guard device comprising:
a funnel-shaped body component having a first end, a hollow interior, and an open second end; and
a small foam insert component which is inserted into the open second end and secured; and
wherein the small foam insert component would be secured within the open second end via gauze tape;
wherein a sacrum wound is cleaned according to medical recommendations then covered with a gauze pad and anti-fungal cream and powder to prevent germs and aid healing;
wherein anus of a user is covered with a gauze pad and anti-fungal cream and powder to help preserve integrity of user's skin in this area;
wherein the funnel-shaped body component is positioned on the gauze pad of the anus;
wherein the first end of the funnel-shaped body component is positioned near the sacrum wound and the open second end and the small foam insert component is positioned by the anus to act as a shield, preventing fecal matter from infiltrating the sacrum wound during its healing process; and
further wherein users can wipe away fecal matter without interrupting a scheduled wound dressing change for the sacrum wound.

15. The fecal guard device of claim 14 further comprising a plurality of indicia.

16. The fecal guard device of claim 14, wherein the fecal guard device is available as a kit.

17. The fecal guard device of claim 16, wherein the kit includes the funnel-shaped body component, multiple gauze pads of varying shapes and sizes, anti-fungal cream and powder, and gauze tape.

18. The fecal guard device of claim 17 wherein the kit is stored in a container with a sterile-packed lid.

19. The fecal guard device of claim 14 wherein the funnel-shaped body component is inserted on the gauze pad over the anus and friction fit between a user's gluteus maximus, during use.

20. A method of preventing fecal matter from infiltrating a stage 4 wound on the sacrum during its healing process, the method comprising the following steps:
providing a fecal guard device comprising a funnel body component with a small foam insert within a hollow opening;
covering anus of a patient with a gauze pad and anti-fungal cream or powder;
clean the sacrum wound according to medical recommendations then covering sacrum wound with a gauze pad and anti-fungal cream or powder;
securing the funnel body component to the gauze pad on the anus via friction fit between a user's gluteus maximus;
expelling fecal matter without worry of the fecal matter touching the sacrum wound; and wiping away fecal matter without interrupting a scheduled sacrum wound dressing change.
